(19) United States
(12) Patent Application Publication
(10) Pub. No.: US 2022/0126003 A1
(43) Pub. Date: Apr. 28, 2022

(54) SIMULATION MODEL FOR SIMULATING A TUBE LINE FOR A BLOOD LEAKAGE DETECTOR, AND METHOD FOR TESTING A BLOOD LEAKAGE DETECTOR

(71) Applicant: Fresenius Medical Care Deutschland GmbH, Bad Homburg (DE)

(72) Inventors: Lena HAHN, Bonn (DE); Alexander SCHRÖRS, Frankfurt a. M. (DE); Sven Marten CZERWONKA, Frankfurta.M. (DE)

(73) Assignee: Fresenius Medical Care Deutschland GmbH, Bad Homburg (DE)

(21) Appl. No.: 17/429,042

(22) PCT Filed: Feb. 3, 2020
(86) PCT No.: PCT/EP2020/052564

§ 371 Date: Aug. 6, 2021

(30) Foreign Application Priority Data
Feb. 7, 2019 (DE) 10 2019 103 028.5

(51) Int. Cl. A61M 1/16; G09B 23/28

(52) U.S. Cl. CPC A61M 1/1692; A61M 1/1613; G09B 23/28; A61M 2205/3306; A61M 2205/587; A61M 2205/70; A61M 2205/15

(57) ABSTRACT

The present invention relates to a simulation model for simulating a tube line comprising at least one hollow cylindrical section that is configured to be arranged in a tube receiver of a blood leak detector of a blood treatment machine, wherein a respective receiver for a respective light source is provided at the axial ends of the hollow cylindrical section, by means of which light source light can be introduced into the hollow cylindrical section; and at least one light guidance element that is arranged in the hollow cylindrical section and that is configured to deflect light introduced into the hollow cylindrical section such that the light moves outwardly out of the hollow cylindrical section through at least one opening in a radial outer side of the hollow cylindrical section. The invention further relates to a method of checking the function of a blood leak detector by means of a simulation model in accordance with the invention.

The present invention relates to a simulation model for simulating a tube line for checking the function of a blood leak detector of a blood treatment machine.

Modern blood treatment machines typically have different monitoring elements that monitor the function of the blood treatment machines before and during a treatment. One of these monitoring elements is the blood leak detector that monitors the filtrate circuit or the plasma circuit of the blood treatment machine for blood leaks.

A blood leak is present as soon as the blood components that should not be removed from the blood via the dialyzer during the treatment are present in the filtrate circuit or in the plasma circuit. This occurs, for example, due to a rupture of the filter membranes in the dialyzer, due to a filter suction, or due to blood flows set too high. Such a blood loss can represent a great risk to the patient if undiscovered.

To avoid this or to recognize it as early as possible, an optical measurement method for detecting a clouding or a red coloration in the filtrate or plasma is carried out by means of the blood leak detector during the operation of the blood treatment machine. In this process, the fact is used for the optical detection of hemoglobin in the filtrate or plasma that the absorption curve of the oxygen-deficient and of the oxygen-rich hemoglobin in the absorption spectrum of hemoglobin in the visible spectral range falls dramatically in the red wavelength range and is thus a lot lower than in the green wavelength range between 500 nm and 600 nm.

The blood leak detector is typically arranged at a tube line and transmits light of a defined wavelength through the tube and thus through the fluid (for example filtrate or plasma) flowing in the tube to a light detector that is typically a phototransistor. The blood leak detector additionally typically has a second light detector or phototransistor that directly receives the light and delivers a reference value. Whether the fluid contains hemoglobin can be determined from the absorption behavior of the fluid or from the corresponding measurement values of the phototransistor.

Each of the two phototransistors delivers two values within a measurement cycle of the blood leak detector that typically lasts a total of 400 ms. Four measurement values result from this (in the following: measurement value red; measurement value green; reference value green; and reference value red) from which a Value and a dimming value can be calculated that reflect the absorption behavior of a solution. The reference values here should be as independent as possible from external influences and should serve, for example, to eliminate aging phenomena of the light source that is typically an LED through the measurement of the direct irradiation from the signals calculated later.

With a normal blood leak, the clouding in the tube due to blood cells is still very low and the Value signal that is calculated using the formula below is used for the monitoring.

$$Value = \frac{\frac{Measurement value red * Reference value green}{Measurement value green * Reference value read}}{\frac{\begin{matrix}Calibration measurement value red * \\ Calibration reference value green\end{matrix}}{\begin{matrix}Calibration measurement value green * \\ Calibration reference value red\end{matrix}}}$$

This signal is composed of both the values of the red light and the values of the green light. As previously mentioned, a greater absorption of the green light occurs due to the hemoglobin, whereby the Value increases accordingly with a normal blood leak.

With massive blood leaks, such great cloudiness occurs in the tube that full absorption of light can occur, whereby the Value is of no use for the detection of massive blood leaks since both red light and green light are absorbed by the great cloudiness. The dimming value that can be calculated only from the values of the red light in accordance with the formula below is therefore used for the recognition of massive blood leaks.

$$Dimming = \frac{Measurement value red * Calibration r eference value red}{Reference value red * Calibration measurement value red}$$

In addition to the current values delivered by the phototransistors, the calibration values are also used for the calculation of the Value and of the dimming value. They are likewise four values that are recorded during a calibration and that are subsequently stored as constant values. A tube filled with saline solution such as a sodium chloride (NaCl) solution is placed into the blood leak detector in the calibration.

To ensure patient safety, the function of the blood leak detector of a blood treatment machine also has to be checked regularly. Conventionally, the blood leak detector is manually tested here in that different defined test solutions such as saline solution, plasma, filtrate, etc. are conducted through the tube line at which the blood leak detector is arranged.

This test method is, however, very complex, slow, and also requires a relatively large amount of resources due to the tubes and solutions required. In addition, the conventional test method is limited to specific test conditions that are e.g. produced by saline solution, plasma, or filtrate in the tube line.

It is thus the underlying object of the present invention to alleviate or remedy the disadvantages of the prior art. It is in particular the underlying object of the present invention to provide a faster test method that requires less resources and to provide an apparatus for carrying out such a test method.

This object is achieved by the simulation model in accordance with claim 1 and by the method in accordance with claim 10. A further aspect of the present invention relates to a blood treatment machine, in particular to a dialysis machine, using a simulation model in accordance with claim 1.

A simulation model in accordance with the invention makes it possible that different test conditions such as filtrate in the tube line or saline solution in the tube line can be simulated by means of a single component without actual solutions or tube lines being used. The function of the blood leak detector can be monitored in an automated and/or (fully) automatic manner using such a simulation model.

A simulation model in accordance with the invention for simulating a tube line comprises: at least one hollow cylindrical section that is configured to be arranged in a tube receiver of a blood leak detector, preferably of a blood leak detector of a blood treatment machine, wherein a respective receiver for a respective light source is provided at the axial ends of the hollow cylindrical section, by means of which light source light can be introduced into the hollow cylindrical section; and at least one light guidance element that is arranged in the hollow cylindrical section and that is configured to deflect light introduced into the hollow cylindrical section such that the light moves to outwardly out of the hollow cylindrical section through at least one opening in a radial outer side of the hollow cylindrical section.

The hollow cylindrical section of the simulation model here preferably simulates a tube line that would conventionally be placed into the tube receiver of the blood leak detector.

Light having a predetermined red portion and/or green portion can be introduced into the hollow cylindrical section by means of the light sources so that the absorption behavior of a solution such as saline solution or of plasma or of any other desired solution or of a fluid can be simulated. Different solutions can be simulated/emulated by the setting of the mixture of the light.

The light sources can be permanently installed in the receivers of the simulation model, preferably by pressing or by adhesive bonding. Alternatively to this, the light sources can also be arranged at a blood treatment machine and/or can be associated with the blood treatment machine and can only be introduced into the receivers of the simulation model when the simulation model is used, for example, to check the function of a blood leak detector of the blood treatment machine.

It has proved to be advantageous if the light guidance element is a mirror that is arranged such that it refracts the introduced light and deflects it by approximately 90°. The light introduced by the light sources is deflected by the light guidance element through the at least one opening in the hollow cylindrical section of the simulation model to at least one light detector such as a phototransistor.

It has further proved to be advantageous if two openings are provided in the radial outer side of the hollow cylindrical section that are preferably arranged opposite one another and between which the at least one light guidance element is preferably arranged.

In accordance with an aspect of the present invention, a respective light source that preferably introduces red and/or green light into the hollow cylindrical section is arranged in the receivers at the axial ends of the hollow cylindrical section. Only one light source can, however, also be provided at or in the simulation model. In addition, the light source can emit light of any other desired wavelength, for example in the visible range or in the non-visible range, in the infrared range, or in the UV range.

The light source is preferably an LED that is firmly anchored in the receiver, preferably through a press fit and/or by adhesive bonding.

To increase the user friendliness of the simulation model and to ensure a secure anchorage of the simulation model at a blood leak detector, the simulation model can additionally be equipped with a handle section that is provided to facilitate a gripping of the simulation model by a user and that preferably extends in arc form or hoop form from a first axial end section of the hollow cylindrical section to a second axial end section of the hollow cylindrical section.

It has further proved to be of advantage if the simulation model is produced from a material, preferably from metal and/or plastic, that is opaque/impermeable for light introduced into the hollow cylindrical section and for environmental light. The simulation model can be produced by means of an additive or subtractive 3D printing process. The material of the simulation model is preferably colored as dark, for example black. A particularly good shielding of the inner space of the hollow cylindrical section from interfering environmental light is hereby achieved.

The simulation model can furthermore additionally have a control device that controls or regulates the at least one light source or the light sources of the simulation model. The mixture (for example the green portion and/or the red portion) of the light transmitted by the at least one light source or by the light sources can, for example, be varied or set by means of the control device.

A further aspect of the invention relates to a blood treatment machine having a simulation model in accordance with the invention and having a blood leak detector that has at least one light detector, wherein the at least one opening in the radial outer side of the hollow cylindrical section of the simulation model and the at least one light detector of the blood leak detector are arranged relative to one another such that light introduced into the hollow cylindrical section and deflected by the light guidance element is directed to the at least one light detector through the at least one opening. The light detector is preferably a phototransistor.

The simulation model can be releasably or non-releasably connected to the blood treatment machine.

Another aspect of the invention relates to a method of checking the function of a blood leak detector of a blood treatment machine and/or the function of a blood treatment machine in which a simulation model is arranged at the blood leak detector, wherein the hollow cylindrical section of the simulation model is preferably arranged in a tube receiver of the blood leak detector.

Light having a predetermined red portion and/or green portion is introduced into the hollow cylindrical section of the simulation model by means of the at least one light source of the simulation model, with the red portion and/or green portion of the light introduced by the light source being set such that it corresponds to the red portion and/or green portion of light that is reflected when, while either no fluid or a specific fluid (such as saline solution, plasma, or filtrate) flows in a tube arranged in the tube receiver, light of a predetermined wavelength is transmitted in or through the tube and/or the fluid. The mixture of the light thus emulates the absorption behavior of a specific solution or simulates it.

The red portion and/or the green portion of the light introduced by the light source is preferably set such that it corresponds to the red portion and/or the green portion of light that is reflected when, while saline solution or plasma or dialysis fluid or plasma having a specific blood portion flows in the tube arranged in the tube receiver and light of a specific wavelength is transmitted in or through the tube and/or the fluid. The conventional test conditions (saline solution, plasma, dialysis fluid, plasma having a specific blood portion) can thus be simulated by means of the simulation model. However, any other desired test conditions can also be simulated by means of the simulation model or simulation method since light of any desired wavelength and/or composition can be used to simulate any desired absorption behavior.

The expected dimming values and Values are known for different predefined states or test conditions. These states are, for example: tube filled with NaCl solution placed in the blood leak detector; normal blood leak in the plasma, for example; massive blood leak, for example in the plasma; no tube placed in the blood leak detector; empty tube placed in the blood leak detector; hemolysis, for example in the plasma; infringement of the valid value ranges; air bubbles in the tube; extraneous light effect on the blood leak detector; simulation of the TO test (initial test when putting a blood treatment machine into operation).

The measurement values of the red and green light (measurement value red and measurement value green in the following) that characterize a specific state are in particular calculated in accordance with the formula below and are then set by the light sources or LEDs in the simulation model, for example with the aid of a digital to analog converter and produce Values and dimming values associated with a specific state.

$$Measurement value red = \frac{\begin{matrix}Dimming * Reference value red * \\ Calibration measurement value red\end{matrix}}{Calibration reference value red}$$

$$Measurement value green = \frac{\begin{matrix}Measurement value red * Reference value green * \\ Calibration measurement value green * \\ Calibration reference value red\end{matrix}}{\begin{matrix}Calibration measurement value red * Reference value red * \\ Calibration measurement value green * Value\end{matrix}}$$

The states or test conditions can be simulated in the simulation model in this manner.

The blood treatment machine can furthermore additionally have a control device that controls or regulates the at least one light source or the light sources of the simulation model. The mixture (for example the green portion and/or the red portion) of the light transmitted by the at least one light source or by the light sources can, for example, be varied or set by means of the control device.

A method in accordance with the invention can comprise the following steps: connecting a simulation model in accordance with the invention to a control apparatus, in particular when the simulation model does not itself have any control apparatus; optionally connecting the simulation model to a test control device that is configured for carrying out a test and by means of which a user can, for example, specify a specific test condition; arranging the simulation model at or in a blood leak detector; synchronizing the frequency of the illumination cycle of the at least one light source (or of the light sources, typically LEDs) with the recording frequency or measurement frequency of the blood leak detector, preferably by optical cycle determination or by reading a DB variable; and transmitting light from the at least one light source (or from the light sources, typically LEDs) that is conducted to the at least one light detector (or to light detectors, typically phototransistors), with the desired measurement values or reference values being reached to emulate or simulate the specified test condition.

DB variables are variables that can be transmitted from the blood treatment machine to a Windows computer via a CAN adapter. They can be manipulated in that values are assigned to them via a script or via the so-called DB tool to make the blood treatment machine believe that states are present that do not really apply at all.

A method in accordance with the invention can be used to test the function of the blood leak detector itself or to check the blood treatment machine for the reactions of the blood treatment machine to different (alarm) states in the blood leak detector.

It is pointed out at this point that the terms “a” and “one” do not necessarily refer to exactly one of the elements, even though this represents a possible embodiment, but can also designate a plurality of elements. The use of the plural equally also includes the presence of the element in question in the singular and, conversely, the singular also includes a plurality of the elements in question.

Further details and advantages of the present invention will be explained in more detail with reference to an embodiment shown in the drawing.

There are shown:

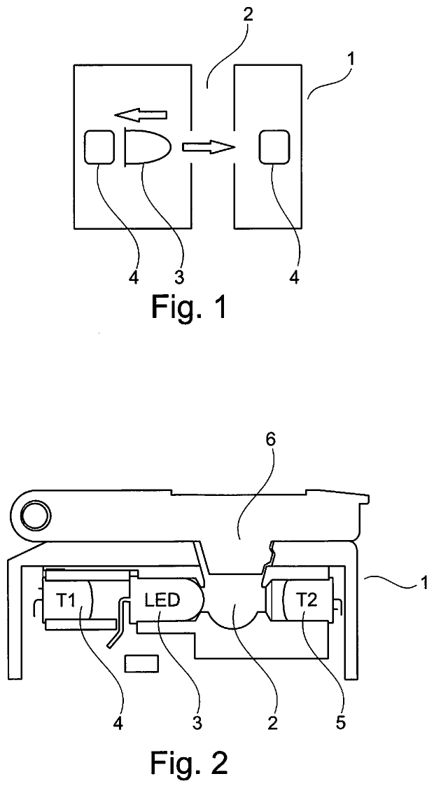

FIG. 1: a schematic view illustrating the function of a blood leak detector;

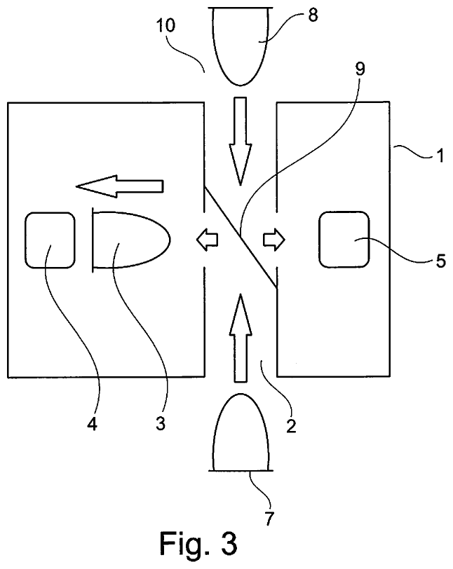

FIG. 2: a sectional view of a blood leak detector;

FIG. 3: a schematic view illustrating the function of a simulation model in accordance with the invention;

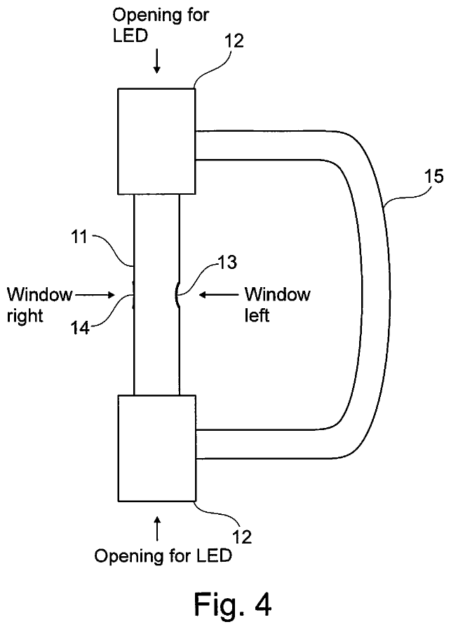

FIG. 4: a plan view of the front side of a simulation model in accordance with a preferred embodiment of the invention;

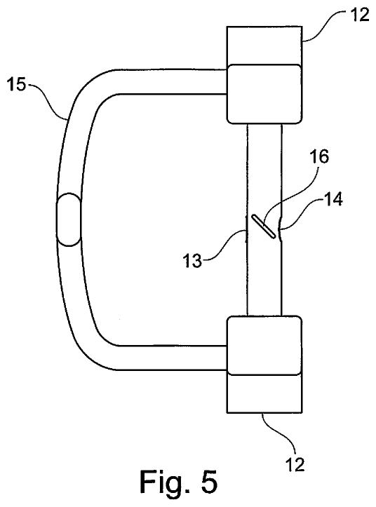

FIG. 5: a plan view of the rear side of a simulation model in accordance with a preferred embodiment of the invention;

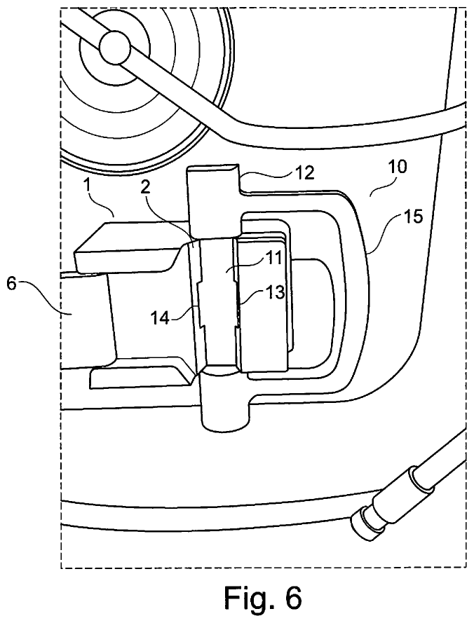

FIG. 6: the simulation model of FIGS. 4 and 5 in the state arranged at a blood leak detector; and

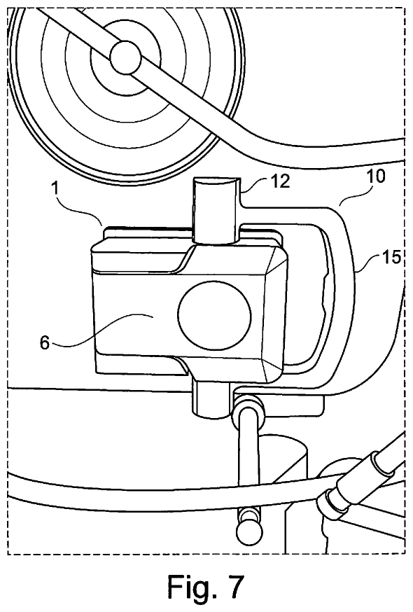

FIG. 7: the simulation model of FIGS. 4 and 5 in the state arranged at a blood leak detector, with the blood leak detector being closed.

The same reference numerals in the Figures designate the same or functionally the same components.

As shown in FIG. 1, a blood leak detector 1 has a tube receiver 2 in which a tube or a tube line can be arranged.

The blood leak detector 1 additionally has an LED 3 and two phototransistors 4 and 5. The light irradiated by the LED 3 is reproduced by arrows in FIG. 1.

Light is transmitted from the LED 3 through the tube receiver 2 and through a tube optionally arranged therein to the phototransistor 4. The phototransistor 4 detects a corresponding measurement value. In addition, light is transmitted directly to the phototransistor 3 by the LED 3. The phototransistor 3 detects a corresponding reference value.

As can be recognized from the sectional view of a blood leak detector in FIG. 2, the blood leak detector 1 furthermore has a door or flap 6 that can be closed after the placing of a tube into the tube receiver 2 so that the tube is firmly held in the tube receiver 2.

FIG. 3 schematically shows a simulation model 10 that is arranged in a blood leak detector 1 and that has two LEDs 7 and 8 and a mirror 9. The hollow cylindrical section, not shown here, of the simulation model is arranged in the tube receiver 2.

Light (shown by the arrows) introduced into the hollow cylindrical section of the simulation model by the LEDs 7 and 8 is deflected substantially at a right angle by the mirror 9 and is directed to the phototransistors 4 and 5 of the blood leak detector.

FIG. 4 shows a plan view of the front side of a simulation model 10. The simulation model has a hollow cylindrical section 11 at whose axial ends respective receivers 12 are arranged to receive the LEDs 7 and 8. Two mutually oppositely disposed openings 13, 14 are formed in the radial outer side of the hollow cylindrical section and light can move outwardly from the hollow cylindrical section 11 through them.

The simulation model 10 further has a hoop-shaped handle section 15 that extends from one receiver 12 to the other receiver 12.

The mirror 16 can be recognized in FIG. 5 that is arranged between the opening 13 and the opening 14 such that light is substantially deflected by 90° and is conducted through the openings 13 and 14 respectively.

In the embodiment shown, the simulation model 10 is produced in one piece by an additive 3D printing process from a black material.

FIG. 6 shows the simulation model 10 that is arranged at the blood leak detector 1. The door 6 of the blood leak detector 1 is open so that the hollow cylindrical section 11 arranged in the tube receiver 2 can be recognized.

In the representation in FIG. 7, the door 6 of the blood leak detector 1 is closed so that the hollow cylindrical section 11 arranged in the tube receiver 2 is firmly held in the blood leak detector.

1. A simulation model for simulating a tube line comprising at least one hollow cylindrical section that is configured to be arranged in a tube receiver of a blood leak detector, preferably of a blood leak detector of a blood treatment machine, wherein a respective receiver for a respective light source is provided at the axial ends of the hollow cylindrical section, by means of which light source light can be introduced into the hollow cylindrical section; and at least one light guidance element that is arranged in the hollow cylindrical section and that is configured to deflect light introduced into the hollow cylindrical section such that the light moves outwardly out of the hollow cylindrical section through at least one opening in a radial outer side of the hollow cylindrical section.

2. A simulation model in accordance with claim 1, characterized in that the light guidance element is a mirror that is arranged such that it deflects the introduced light by approximately 90°.

3. A simulation model in accordance with claim 1, characterized in that two openings are provided in the radial outer side of the hollow cylindrical section that are preferably disposed opposite one another and between which the at least one light guidance element is preferably arranged.

4. A simulation model in accordance with claim 1, characterized in that a respective light source that preferably introduces red and/or green light into the hollow cylindrical section is arranged in the receivers at the axial ends of the hollow cylindrical section.

5. A simulation model in accordance with claim 4, characterized in that the light source is an LED that is firmly anchored in the receiver, preferably by a press fit and/or by an adhesive bond.

6. A simulation model in accordance with claim 1, additionally comprising a handle section that is provided to facilitate a gripping of the simulation model by a user and that preferably extends in arc-like form or hoop-like form from a first axial end section of the hollow cylindrical section to a second axial end section of the hollow cylindrical section.

7. A simulation model in accordance with claim 1, characterized in that the simulation model is produced from a material, preferably from metal and/or plastic, that is opaque for light introduced into the hollow cylindrical section and for environmental light.

8. A blood treatment machine having a simulation model in accordance with claim 1 and a blood leak detector that has at least one light detector, characterized in that the at least one opening in the radial outer side of the hollow cylindrical section of the simulation model and the at least one light detector of the blood leak detector are arranged such that light deflected by the light guidance element and introduced into the hollow cylindrical section is conducted through the at least one opening to the at least one light detector.

9. A blood treatment machine in accordance with claim 8, characterized in that the simulation model is releasably or non-releasably connected to the blood treatment machine.

10. A method of checking the function of a blood leak detector of a blood treatment machine comprising the step: arranging a simulation model in accordance with claim 1 at the blood leak detector, preferably arranging the hollow cylindrical section of the simulation model in a tube receiver of the blood leak detector.

11. A method in accordance with claim 10, further comprising the step: introducing light having a predetermined red portion and/or green portion into the hollow cylindrical section of the simulation model by means of the at least one light source, with the red portion and/or green portion of the light introduced by the light source being set such that it corresponds to the red portion and/or green portion of light that is reflected when, while either no fluid or a specific fluid flows in a tube arranged in the tube receiver, light of a predetermined wavelength is transmitted in or through the tube and/or the fluid.

12. A method in accordance with claim 11, characterized in that the red portion and/or the green portion of the light introduced by the light source is set such that it corresponds to the red portion and/or the green portion of light that is reflected when, while saline solution or plasma or dialysis fluid or plasma having a specific blood portion flows in the tube arranged in the tube receiver and light of a specific wavelength is transmitted in or through the tube and/or the fluid.
